(19) United States
(12) Patent Application Publication
(10) Pub. No.: US 2015/0298006 A1
(43) Pub. Date: Oct. 22, 2015

(54) HELIX PROFILE SYSTEM AND METHODS

(71) Applicant: DNA Diagnostics Center, Inc., Fairfield, OH (US)

(72) Inventors: David Silver, Cincinnati, OH (US); Michael L. Baird, West Chester, OH (US); Richard Chmelo, West Chester, OH (US); Russ Lamkin, Cincinnati, OH (US); Megan Gossard, Cincinnati, OH (US); Joe Kromer, Cincinnati, OH (US)

(21) Appl. No.: 14/693,613

(22) Filed: Apr. 22, 2015

Related U.S. Application Data
(60) Provisional application No. 61/982,500, filed on Apr. 22, 2014.

(51) Int. Cl. A63F 13/65; C12Q 1/68

(52) U.S. Cl. CPC A63F 13/65; C12Q 1/6876; C12Q 2600/158; C12Q 2600/124

(57) ABSTRACT

A system and method for developing an avatar personalized from a user's own DNA, where the avatar can borrow or include additional traits from other avatars. The avatar may be used in a variety of platforms, including games, profiles, etc. An avatar may be created from the DNA of others, such as a celebrity avatar. Examples of traits derived from a helix profile include, but are not limited to: Strength, Speed, Health, and Activity.

### CROSS-REFERENCE TO RELATED APPLICATION

This application claims the benefit of U.S. Provisional Patent Application Ser. No. 61/982,500, filed Apr. 22, 2014, entitled “HELIX PROFILE SYSTEM AND METHODS,” the entirety of which is incorporated herein by reference.

### FIELD OF THE INVENTION

The present subject matter relates to creating a personalized avatar or environment from a helix profile derived from a user's DNA (deoxyribonucleic acid). Video games may employ the use of an avatar or environment (heretofore referred to as avatar) based from a user's own helix profile or one created by another user, for example, a celebrity user. A helix profile contains identifying markers that may include, but is not limited to, information regarding attributes, such as speed, strength, and size.

### BACKGROUND

Avatar creation has consisted of programmed software that specifies traits of an avatar. The avatar or traits may correspond to physical features that represent an avatar. The values for each trait are set by selecting from a list of options or entering traits directly (e.g. hair color, weight, etc.). User traits may be stored on the user's local computer and/or a network.

Avatars that are linked by inheritance are linked only to other generated characters in the game. Randomly generated avatars may be used to create an initial model and then a user may alter the avatar as desired by manually entering the appropriate changes. Software products that allow the selection of an avatar and/or manipulation of an avatar model are limited in that the features of the avatar are not based off the user's own DNA.

### SUMMARY

A method and system for generating at least one avatar, comprising the steps of receiving DNA testing information from a user, developing an individualized helix profile based on the DNA testing information, and generating an avatar based on the helix profile and related algorithm to convert DNA test results. An individualized helix profile may be correlated to predetermined character traits that are embodied by the avatar. An avatar may be generated based on a combination of the individualized helix profile and user-selected traits, or a combination of at least two or more avatars, wherein additional character traits are either permanently or temporarily included in the avatar. An avatar can be used cross-functionally in more than one virtual environment, game, etc. Examples of traits derived from a helix profile include, but are not limited to: Strength, Speed, Health, Customized Weapons, Activity, specific Athletic Abilities, specific Mystical Abilities, etc.

### DETAILED DESCRIPTION

Reference will now be made in detail to exemplary embodiments of the present teachings, examples of which are illustrated in the accompanying drawings. It is to be understood that other embodiments may be utilized and structural and functional changes may be made without departing from the respective scope of the present teachings. Moreover, features of the various embodiments may be combined or altered without departing from the scope of the present teachings. As such, the following description is presented by way of illustration only and should not limit in any way the various alternatives and modifications that may be made to the illustrated embodiments and still be within the spirit and scope of the present teachings.

Recording and Analyzing Data for Helix Profiling

To obtain DNA information of a user, a user may utilize an in-home DNA testing kit. The DNA testing kit may include collecting a DNA sample, for example, by swabbing the inside of the cheek, and mailing the sample to a laboratory. In an embodiment, a DNA testing kit may be coupled with a video game purchase. In another embodiment, a DNA testing kit may be available separately by an in-store, mail purchase, trade show, event, etc. Various ways that a DNA sample can be collected may include a swab of saliva, sample of hair, sample of blood, etc. Other known and suitable DNA samples may be tested. DNA information may be collected via DNA ancestry test results, which recreate a world from the player's ancestry.

Users may be notified regarding their DNA helix profile by mail, phone, email, online, etc. In an embodiment, the results of the DNA helix profile are loaded directly into a user's video game profile to create an avatar. A DNA helix profile can then be authorized for use and/or sharing for a customized avatar. If the user does not wish to use or share their avatar publically, they may elect to do so and their customized helix profile will be removed from the database. On the other hand, if a user wishes to maintain their helix profile avatar, then the user may also share their avatar with others. For example, celebrity users may wish to create an avatar based on their DNA helix profile, for others to use.

Specimen and test results may be saved at appropriate storage facilitates (e.g. diagnostic testing company, gaming company, laboratory, third party, etc.). The conditions may include, for example, a temperature controlled setting. In one embodiment, the storage conditions may be set at a relatively constant room temperature with no higher than 20% humidity. Electronic data may be stored in a secure server and/or storage facility with limited access and multiple backs-ups by appropriate parties. It may be useful to save specimen and test results for future upgrades regarding the number of markers measured, the types of traits tests, the evolvement of the accuracy available in testing, etc. for developing a helix profile.

In an embodiment, a person may use their own DNA to generate a helix profile for an avatar that serves as a representation of the user for use online, in video games, virtual worlds, social media, electronic profiles, online communities, chat rooms, medical records, etc. The user may be any entity that could have DNA (e.g. human, animal, etc.) and any entity that could theoretically have DNA (e.g. ogre, mascot, mystical creature). In an embodiment, a helix profile avatar may be shown as an icon.

Markers

At least one marker may be tested and/or selected from a user's DNA test results to create an avatar. A test may be offered including a specific number of DNA markers, such as STR DNA markers, in which all or some of the markers are subsequently chosen for creation of an avatar.

In one embodiment, up to twenty-two unique identifying markers, including speed, strength, and size, are translatable into a user's individualized helix profile avatar. A helix profile avatar may represent physical features, such as hair color, eye color, skin color, etc. In an embodiment, a helix profile avatar may represent traits that are not readily seen from the outward appearance of a user. In an embodiment, a helix profile may solely include traits relating to speed, strength, and size.

In additional embodiments, up to approximately five, ten, twenty, thirty, forty, fifty, sixty, seventy, eighty, ninety, one hundred, one hundred and fifty, two hundred, two hundred and fifty, three hundred, three hundred and fifty, four hundred, four hundred and fifty, or five hundred unique identifying markers may be selected. In an embodiment, any suitable number of markers may be selected and utilized in creation of the avatar, based on user selection. In an embodiment, a default number of markers may be offered, although a greater number of markers may be tested. As a user progresses in a game or achieves an upgrade in a game, additional traits derived from additional markers may be revealed.

In an embodiment, markers can be chosen to represent a user's behaviors, sounds, gestures, motions, real age, etc. A marker may be used to test for traits that are translatable into useful avatar traits, such as Strength, Speed, Health, Activity, etc.

Various exemplary embodiments of genetic traits that may be tested and translated into useful avatar traits include, but are not limited to: Strength: bone density, immune response, muscle tone, muscle cramps, slow twitch muscles, muscle mass, throwing distance, shooting distance, striking distance, explosive power; Speed: swiftness, jumping ability, fast twitch muscles, quickness, reaction time, throwing speed; Endurance: distance, stamina and length of time performing an activity, repetition ability, recovery time; Health: bleeding/circulation, breathing (or holding breath), vision, appetite, hearing, taste, smell, touch, pain tolerance, recovery from injury; Movement: energy metabolism, muscle repair, activity level, coordination, balance; Target and Attack: ability to generate and target opposing video game characters with at least one of fire, water, ice, radiation, electricity, sound, vibrations, energy, and physical weapons; Personality: shyness, intelligence, level of fright, aggressive behavior, quietness, calmness, ability to work within a team; Sensitivity detecting and/or measuring the following: vibration, brain waves, gravity, radiation including infrared/gamma rays, light waves, sound, energy, electricity, heat, cold, odor, humidity, radar; Focus: attention to detail, patience, accuracy, aim, multi-tasking; etc. Any one or more of these traits or other suitable genetic traits may be tested and translated into useful avatar traits.

In an embodiment, once an initial set of markers is chosen, a subsequent group of marker may be integrated relating to additional maker results. In other words, as a user's avatar is involved in a game, an initial set of markers form an initial DNA-based profile, however, as the user proceeds through the game, additional markers are unlocked or added to the avatar's initial profile to create a more robust profile over time.

Manipulation of Information from DNA into Avatar Form

DNA testing is useful in yielding information regarding a user's speed, strength, and size.

In an embodiment, as shown in Table 1, the below traits are evaluated and assessed a corresponding value, attribute, relativity, and ranking, in developing a helix profile avatar:

TABLE 1

| Genetic | | | | |
|---|---|---|---|---|
| Markers | Value | Attribute | Relativity | Ranking |
| TPOX | 13 | Strength | Average | 52% |
| TH01 | 17 | Intelligence | Above Average | 65% |
| D21S11 | 14 | Speed | Superior | 93% |

As shown above in Table 1, TPOX, TH01, and D21S11 refer to examples of genetic markers.

In an embodiment, a specified model may be utilized for predicting certain personality traits to be included in the helix profile. For example, a Myers-Briggs Type Indicator® (MBTI®) may be used with or without DNA test results to provide a more robust offering, by linking certain test results with identified personality traits. In an embodiment, an image may be formed to encapsulate the DNA test results and attributes (e.g. Strength, Speed, Agility, Intelligence, etc.) represented by a specific user. This image may be customizable for each game, partnership, level of play, etc. In another embodiment, a gene, for example the MAO-A (“Warrior gene”), may be tested and specified traits may be linked with the results from the test. These test results may further be correlated to a trait and be scaled with results found across any suitable population, such as the general population.

In an exemplary embodiment, select markers and/or genes may be correlated to key attributes that can be utilized in a game, for example. The identification of genes that relate to the following key attributes can be useful for key game attributes: Strength, Speed, Health, and Activity.

As shown in Table 2, an example of a game attribute is correlated with specific genes, which may further include a scaling variability or ranking based on the frequency of each specific gene in the general population, and optionally, bonus improvements are afforded to a user:

TABLE 2

| | | | | | Example of |
|---|---|---|---|---|---|
| | | | | | Bonus |
| | | | | | Improvements |
| | | | | | in Game |
| | | | | | (based on |
| | | | Example | Example of | desired |
| Identified | | | of | Frequency | results) |
| Game | | Potential | Desirable | in General | (e.g. %, |
| Attribute | Gene | Outcomes | Results | Population | points, etc.) |
| Strength | Gene 1 | AA, Aa, | aa | 20% | 50% increase |
| | | aa | | | |
| Strength | Gene 2 | BB, Bb, | BB | 10% | 75 point |
| | | bb | | | increase |
| Strength | Gene 3 | cc, cC, | cc | 30% | 3 second |
| | | CC | | | temporary |
| | | | | | increase |
| Strength | Gene 4 | DD, Dd, | DD | 10% | 10% increase |
| | | dd | | | |
| Speed | Gene 5 | EE, Ee, | ee | 20% | 5% |
| | | ee | | | improvement |
| Speed | Gene 6 | ff, fF, | FF | 30% | 5% |
| | | FF | | | improvement |
| Speed | Gene 7 | GG, Gg, | gg | 20% | 5% |
| | | gg | | | improvement |
| Health | Gene 8 | hh, hH, | HH | 20% | 3 second |
| | | HH | | | longevity |
| | | | | | increase |
| Health | Gene 9 | II, Ii, ii | ii | 60% | 4 second |
| | | | | | longevity |
| | | | | | increase |
| Health | Gene 10 | jj, jJ, JJ | JJ | 40% | 7 second |
| | | | | | longevity |
| | | | | | increase |
| Health | Gene 11 | KK, Kk, | KK | 50% | 10 second |
| | | kk | | | longevity |
| | | | | | increase |
| Activity | Gene 12 | ll, lL, LL | ll | 70% | 10 second |
| | | | | | activity |
| | | | | | increase |
| Activity | Gene 13 | MM, Mm, | MM | 60% | 30 point |
| | | mm | | | increase in |
| | | | | | activity level |
| Activity | Gene 14 | nn, nN, | nn | 10% | 5% increase |
| | | NN | | | |

Based on Table 2, each of the genes tested are assigned to specific information that is correlated to an application for a character trait, as shown below in Table 3.

TABLE 3

| Identified | | | |
|---|---|---|---|
| Game | | Information Tied to | Application for a |
| Attribute | Gene | Each Gene Measured | Character Trait |
| Strength | Gene 1 | Bone Density Potential | These genes are tied |
| Strength | Gene 2 | Bone Density Potential | to an individuals' bone |
| | | | mass density and the |
| | | | strength of the bones |
| | | | within their body. |
| Strength | Gene 3 | Immune Response from | This gene correlates to an |
| | | Infection or from Trauma | individual's immune |
| | | | response from infection |
| | | | or trauma. |
| Strength | Gene 4 | Behavioral Aggression | This gene is tied to an |
| | | | individual's behavioral |
| | | | aggression in response to a |
| | | | provocation or attack. |
| Speed | Gene 5 | Endurance and Speed | This gene is tied to an |
| Speed | Gene 6 | | individual's explosive |
| | | | strength and speed. |
| Speed | Gene 7 | Skeletal Muscle | This gene is tied to an |
| | | Metabolic Efficiency for | individual's endurance as it |
| | | Pulmonary Muscle Tone | relates to muscle tone. |
| Health | Gene 8 | Oxidative Stress | This gene is tied to an |
| | | | individual's ability to |
| | | | prevent oxidative stress |
| | | | and keep their general |
| | | | health. |
| Health | Gene 9 | Oxidative Damage and | This gene is tied to an |
| | | Stress Protection | individual's ability to |
| | | | protect cells from oxidative |
| | | | stress and bacteria. |
| Health | Gene 10 | Vitamin Processing and | These genes are tied to an |
| Health | Gene 11 | Protein Development | individual's energy |
| | | | production and general |
| | | | nutritional health and |
| | | | thus the avatar's Health |
| | | | traits. |
| Activity | Gene 12 | Energy Metabolism | These genes are tied to an |
| Activity | Gene 13 | Regulation | individual's ability to |
| | | | regulate and utilize energy. |
| Activity | Gene 14 | Muscle Repair and | This gene is tied to an |
| | | Regeneration | individual's ability to |
| | | | ensure repair and |
| | | | regeneration of its muscles. |

Table 3 describes embodiments in which genes are selected for testing, then linked to information relating to the user, and finally correlated to performance in the avatar.

In an embodiment, the Strength traits are formed based on at least one of the following: Bone Density Potential; Immune Response from Infection; Immune Response from Trauma; and Behavioral Aggression.

In an embodiment, Speed traits are formed based on at least one of the following: Endurance; Speed; and Skeletal Muscle Metabolic Efficiency for Pulmonary Muscle Tone.

In an embodiment, Health traits may be formed based on at least one of the following: Oxidative Stress; Oxidative Damage; Physical Damage; Stress Protection; Vitamin Processing; and Protein Development.

In an embodiment, Activity traits may be formed based on at least one of the following: Energy Metabolism; Muscle Repair; Regeneration; Tone and Cramping; Pain Tolerance; and Pain Intolerance.

Scaling or ranking of specific character traits may be evaluated based on numerous ranking systems. In one embodiment, the scaling may be based on the current human population in a certain demographic, or the population of avatars in a specific universe, such as a specified video game or in a specific city, state, country, etc. The scaling may be continuously or periodically updated based on the selected demographic and/or population

In another embodiment, the method may include a step whereby the generated helix profile avatar is further manipulated by a user. This will allow a user to create a hybrid of features that is partly based on their own DNA and partly manually selected by the user, or partly based on their own DNA and partly based on the DNA of another user.

Avatar

An avatar is not limited to being a realistic depiction of a user. Instead, an avatar may include a fictionalized depiction of a user, in a human or non-human avatar form. There are many ways that a helix profile can be visually represented, for example, a helix profile may be translated into the form of a fictional character that contains attributes relating to the user, based on their DNA testing results. Additional representations of the avatar may include an animal, mammal, human, monster, chimera, cyborg, god, plant, mystical creature, athlete, solider, insect, fish, bird, inanimate object, machine, car, or robot.

Embodiments of the system and method may include various components of a computing system, which may include software and hardware, such as ROM, RAM, cloud computing, a microprocessor, central servers, memory, keyboards, scanners, consoles, video cameras, network interfaces, etc.

In an embodiment, various groups of avatars may exist forming a classification system of avatars. In one embodiment, the various groups of avatars may include a selection from a group of warriors. For example, a first warrior type may be focused on intelligent aggression against the selected targets at the right time for users interested in destroying enemy guardians, a second warrior type may be focused on in-your-face durability and control abilities for users interested in wading into battle and shaking up enemies, a third warrior type may be focused on changing the outcome of a situation through indirect means, such as blinds or silences, for users who prefer finesse to force, and a fourth warrior type may be focused on the big picture of the battle rather than skirmishes for users who enjoy playing in lane and taking objectives.

In an embodiment, an avatar may further include elective traits selected by a user, such as clothing, shoes, hats, tattoos, jewelry, glasses, and piercings.

In further embodiments, an avatar may defeat another avatar and selectively absorb at least one of another's character traits. Similarly, an avatar may borrow or link up with another avatar's results, such as a celebrity avatar, a friend avatar, a family-related avatar, etc. Although some character traits may be added to an avatar, other traits do not have variations and therefore can only be replaced, not supplemented. For example, a character traits relating to a specific type of animal, such as a German Shepard dog, can only be altered by replacement of another animal type, such as a Labrador dog. However, in other embodiments, the character traits relating to a specific avatar may be mixed with another avatar's traits, for example, a Germany Shepard dog may be mixed with a Labrador dog.

DNA Helix Groups

In an embodiment, avatars that share a common helix profile or any one or a number of DNA markers, may be linked together to form a group, clan, etc. Further, related avatars may be connected through various relationships, such as those based on information relating to family, household, peer groups, organizations, community, social commitments, culture, etc.

Networks of avatars may be created across various gaming and non-gaming profiles, thus allowing the avatar to work on several different virtual environments and games. For example, an avatar with specified traits may be an animal in an ocean-themed game, however the avatar may transform to a human in a tennis-themed game, while retaining each of its personality traits derived from its DNA markers.

User Input

In an embodiment, a virtual world, such a video game, will provide a DNA Selection Tool to allow a user to selectively unlock the use of DNA as attributable to an avatar's traits. The DNA Selection Tool may be opened through clicking on a tab or button, utilizing a specified passcode, completing a specified number of hours of play, completing a specified level of play, solving a puzzle or riddle, achieving membership for a specified time, etc. In an embodiment, a DNA Selection Tool may be accessed only by a user playing the video game, with unique credentials, in order to protect the identity of the user's avatar. A User Feedback Tool may further be utilized so that a user can provide real-time feedback. In an embodiment, a user playing a video game may provide feedback based on whether their current physical abilities, strength, etc. correspond to the same features of the user's avatar. This may allow for analysis and collection of in-depth data as it relates to identifying and correlating DNA results with the user's actual traits.

Although the embodiments of the present teachings have been described in the accompanying embodiments and in the foregoing detailed description, it is to be understood that the present teachings are not to be limited to just the embodiments disclosed, but that the teachings described herein are capable of numerous rearrangements, modifications and substitutions.

We claim:

1. A method for generating at least one avatar, comprising the steps of:
receiving DNA testing information from a user;
developing an individualized helix profile based on the DNA testing information; and
generating an avatar based on the helix profile.

2. The method of claim 1, wherein the individualized helix profile is correlated to predetermined character traits that are embodied by the avatar.

3. The method of claim 2, wherein any of the following genetic traits may be correlated to the predetermined character traits:
Strength: bone density, immune response, muscle tone, muscle cramps, slow twitch muscles, muscle mass, throwing distance, shooting distance, striking distance, explosive power;
Speed: swiftness, jumping ability, fast twitch muscles, quickness, reaction time, throwing speed;
Endurance: distance, stamina and length of time performing an activity, repetition ability, recovery time;
Health: bleeding/circulation, breathing (or holding breath), vision, appetite, hearing, taste, smell, touch, pain tolerance, recovery from injury;
Movement: energy metabolism, muscle repair, activity level, coordination, balance;
Target and Attack: ability to generate and target opposing video game characters with at least one of fire, water, ice, radiation, electricity, sound, vibrations, energy, and physical weapons;
Personality: shyness, intelligence, level of fright, aggressive behavior, quietness, calmness, ability to work within a team;
Sensitivity detecting and/or measuring the following: vibration, brain waves, gravity, radiation including infrared/gamma rays, light waves, sound, energy, electricity, heat, cold, odor, humidity, radar; and
Focus: attention to detail, patience, accuracy, aim, multi-tasking.

4. The method of claim 1, wherein the avatar is generated based on a combination of the individualized helix profile and user-selected traits.

5. The method of claim 4, wherein the avatar is generated based on a combination of at least two or more avatars, wherein additional character traits are either permanently or temporarily included in the avatar.

6. The method of claim 1, wherein the avatar is used cross-functionally in more than one virtual environment.

7. The method of claim 1, wherein the helix profile is developed based on the following traits: Strength, Speed, Health, and Activity.

8. The method of claim 7, wherein the Strength traits are formed based on at least one of the following: Bone Density Potential; Immune Response from Infection;
Immune Response from Trauma; and Behavioral Aggression.

9. The method of claim 7, wherein the Speed traits are formed based on at least one of the following: Endurance; Speed; and Skeletal Muscle Metabolic Efficiency for Pulmonary Muscle Tone.

10. The method of claim 7, wherein the Health traits are formed based on at least one of the following: Oxidative Stress; Oxidative Damage; Physical Damage, Stress Protection; Vitamin Processing; and Protein Development.

11. The method of claim 7, wherein the Activity traits are formed based on at least one of the following: Energy Metabolism; Muscle Repair; Regeneration; Tone and Cramping; Pain Tolerance; and Pain Intolerance.

11. The method of claim 1, wherein the DNA testing information is received by any one or more of the following tests: a swab of saliva, a sample of hair, a sample of blood, a DNA testing kit.

12. The method of claim 1, further comprising modifying the helix profile by at least one of replacing, adding, and mixing traits from another avatar.

13. The method of claim 1, further comprising forming a new avatar based on at least one avatar.

14. The method of claim 1, further comprising transferring information regarding a helix profile to another avatar, virtual environment, storage center, or a combination thereof.

15. The method of claim 1, wherein additional traits are added to an avatar after an initial creation of the avatar.

16. The method of claim 1, wherein at least two or more avatars may be linked by any one or more of the following: family, ancestry, location, relationship, team, and the type of animal, mammal, human, monster, chimera, cyborg, god, plant, mystical creature, athlete, solider, insect, fish, bird, inanimate object, machine, car, and robot.

17. The method of claim 1, further comprising the selection of elective traits, such as clothing, shoes, hats, tattoos, jewelry, glasses, and piercings.

18. The method of claim 1, further comprising including a subsequent DNA test to provide additional DNA testing information.
